(19) United States
(12) Patent Application Publication
(10) Pub. No.: US 2026/0321346 A1
(43) Pub. Date: Oct. 15, 2026

(54) Artificial Intelligence-Based Variant Caller

(71) Applicant: Bristol-Myers Squibb Company, Princeton, NJ (US)

(72) Inventors: Mark David Stern, Cranbury, NJ (US); Daniel Brami, San Diego, CA (US); Gaurav Jain, Bengaluru (IN)

(73) Assignee: Bristol-Myers Squibb Company, Princeton, NJ (US)

(21) Appl. No.: 19/641,774

(22) Filed: Apr. 8, 2026

Related U.S. Application Data
(60) Provisional application No. 63/786,665, filed on Apr. 10, 2025.

(51) Int. Cl. G16B 20/20; G16B 30/10; G16B 40/00

(52) U.S. Cl. CPC G16B 20/20; G16B 30/10; G16B 40/00

(57) ABSTRACT

A method includes receiving a plurality of sequencing data files each obtained by sequencing a nucleic acid sequence from a corresponding biological sample. For each corresponding sequencing data file, the method also includes receiving a corresponding set of variant call format (VCF) files each identifying variants and generated as output from a respective variant caller application that receives the corresponding sequencing data file as input. For each of one or more of the variants by respective VCF file, the method also includes generating a respective pileup image. The method also includes training the neural network model on the pileup images to teach the neural network model to learn how to generate an output identifying variants from sequencing data inputs obtained from sequencing nucleic acid sequences at depths of 10× or less from one or more other biological samples.

### CROSS-REFERENCE TO RELATED APPLICATIONS

This U.S. Patent Application claims priority under 35 U.S.C. § 119(e) to U.S. Provisional Application 63/786,665, filed on Apr. 10, 2025. The disclosure of this prior application is considered part of the disclosure of this application and is hereby incorporated by reference in its entirety.

### TECHNICAL FIELD

This disclosure relates to an artificial-intelligence (AI)-based variant caller and related methods of generating training datasets, training the variant caller, and using the variant caller to call variants.

### BACKGROUND

Next generation sequencing (NGS) technologies such as whole genome sequencing (WGS), whole exome sequencing (WES), and whole transcriptome sequencing (WTS) have become valuable for identifying variants in nucleic acid sequences from samples obtained from patients in clinical settings. However, it is often prohibitively expensive to perform comprehensive WGS, WES, and WTS on biological samples at the standard depth coverages (~30× and above) that are typically required to accurately identify variants, such as single nucleotide variants (SNVs) and short insertions/deletions (inDels). This cost is compounded when large numbers of samples are sequenced.

While performing WGS and WES at low depth coverages (~10× or less) represents a more cost-effective approach for sequencing, this approach generally requires imputation to identify variants, which involves using a separate reference panel for imputing the variants. Such a reference panel consists of a collection of sequences obtained from a population of other people. Reference panels can be biased because they contain predominantly sequences from individuals of certain ancestries, and imputation using such a reference panel may result in failure to identify genetic variants in sequences from individuals of ancestries that differ from the ancestry or ancestries predominantly represented in the reference panel. Accordingly, there is a need for improved methods and systems to call variants, especially rare variants, in sequences from NGS performed at low depth coverages. There is also a need for methods and systems that can call variants without imputation.

### SUMMARY

One aspect of the disclosure provides a computer-implemented method executing on data processing hardware that causes the data processing hardware to perform operations for training a neural network model to learn how to generate output variant calls from sequencing data inputs obtained from sequencing nucleic acid sequences at depths of 10× or less from biological samples. The operations include receiving a plurality of sequencing data files collected from a population of subjects, each sequencing data file obtained from sequencing a nucleic acid sequence from a corresponding biological sample collected from a corresponding subject in the population of subjects. For each corresponding sequencing data file among the plurality of sequencing data files, the operations include receiving a corresponding set of variant call format (VCF) files generated for the corresponding sequencing data file, each respective VCF file in the corresponding set of VCF files generated as output from a respective one of a plurality of independent variant caller applications that receive the corresponding sequencing data file as input, each respective VCF file in the corresponding set of VCF files identifying variants. For each of one or more of the variants identified by each respective VCF file in the corresponding set of VCF files, the operations include generating a respective pileup image. The operations also include training the neural network model on the pileup images generated for the plurality of sequencing data files to teach the neural network model to learn how to generate an output identifying variants from sequencing data inputs obtained from sequencing nucleic acid sequences at depths of 10× or less from one or more other biological samples.

Implementations of the disclosure may include one or more of the following optional features. In some implementations, the corresponding sequencing data file includes a sequence alignment file (SAF) obtained from the sequencing of the nucleic acid sequence from the corresponding biological sample. In some examples, the nucleic acid sequence includes a deoxyribonucleic acid (DNA) sequence or a ribonucleic acid (RNA) sequence. Optionally, the nucleic acid sequence may include a deoxyribonucleic acid (DNA) sequence. The nucleic acid sequence may include a sequence of at least 50 nucleotides. In some implementations, the sequencing of the nucleic acid sequence from the corresponding biological sample includes paired end or single end sequencing. In some examples, the sequencing of the nucleic acid sequence from the corresponding biological sample includes whole genome sequencing or whole exome sequencing.

Optionally, the sequencing of the nucleic acid sequence from at least one of the plurality of biological samples is performed using whole genome sequencing and the sequencing of the nucleic acid sequence from at least another one of the plurality of biological samples is performed using whole exome sequencing. In some implementations, the sequencing of the nucleic acid sequence from the corresponding biological sample includes germline sequencing of the nucleic acid sequence from the corresponding biological sample. In these implementations, the germline sequencing may be performed at a depth of at least 50x. In some examples, the sequencing of the nucleic acid sequence from the corresponding biological sample includes somatic sequencing of the nucleic acid sequence from the corresponding biological sample. In these examples, the somatic sequencing may be performed at a depth of at least 150x.

The plurality of biological samples collected from the population of subjects may include a subset of biological samples collected from a respective subject in the population of subjects. In some implementations, for at least one corresponding biological sample from the subset of biological samples collected from the respective subject, the sequencing of the nucleic acid sequence from the at least one corresponding biological sample includes somatic sequencing of the nucleic acid sequence from the at least one corresponding biological sample from the subset of biological samples collected from the respective subject, and for at least another corresponding biological sample from the subset of biological samples collected from the respective subject, the sequencing of the nucleic acid sequence from the at least another corresponding biological sample includes germline sequencing of the nucleic acid sequence from the at least another corresponding biological sample from the subset of biological samples collected from the respective subject. Optionally, two or more biological samples in the subset of biological samples collected from the respective subject are collected at different timepoints. In some examples, the sequencing of the nucleic acid sequence from the two or more biological samples collected from the respective subject at different timepoints includes somatic sequencing of the nucleic acid sequence from the two or more biological samples. In some implementations, the two or more biological samples collected from the respective subject at different timepoints include tumor biopsy samples. The two or more biological samples collected from the respective subject at different timepoints may include liquid biopsy samples. In these examples, the liquid biopsy samples may include at least one of blood samples, urine samples, sputum samples, or cerebrospinal fluid samples.

In some implementations, the corresponding sequencing data file includes one or more quality control (QC) parameters for one or more loci in the nucleic acid sequence sequenced from the biological samples. Optionally, the corresponding sequencing data file may include one or more QC parameters for each locus in the nucleic acid sequence sequenced from the corresponding biological samples. In some examples, the operations further include, for the variants identified by the corresponding set of VCF files generated for the corresponding sequencing data file, filtering the variants based on the one or more QC parameters of the corresponding sequencing data file to remove any variants in the corresponding set of VCF files that are artifacts. Here, generating the respective pileup image for each of the one or more of the variants identified by each respective VCF file in the corresponding set of VCF files includes generating the respective pileup image for each of the one or more of the variants after filtering to remove the variants in the corresponding set of VCF files that are artifacts.

At least a portion of the biological samples collected from the population of subjects may include biological samples not preserved with a preservative or cross-linking agent. In some implementations, the at least the portion of the biological samples not preserved with the preservative or cross-linking agent include fresh biological samples. In some examples, the biological samples collected from the population of subjects include formalin-fixed paraffin-embedded (FFPE) samples. One or more data augmentation techniques may be performed on the corresponding sequencing data file received as input by the plurality of independent variant caller applications. Optionally, the variants identified by each respective VCF file in the corresponding set of VCF files may include at least one of single nucleotide variants (SNVs), insertions, or deletions.

In some implementations, the operations further include, for at least one corresponding sequencing data file, obtaining a corresponding benchmark data set of variant calls for the nucleic acid sequence sequenced from the corresponding biological sample, and for each respective VCF file in the corresponding set of VCF files generated for the corresponding sequencing data file, annotating, using the benchmark data set of variant calls, the respective pileup image generated for each of the one or more of the variants identified by the respective VCF file to flag any variants correctly identified by the respective VCF file, any variants incorrectly identified by the respective VCF file, and any variants unidentified by the respective VCF file. Here, training the neural network model on the pileup images includes training the neural network on the annotated pileup images. In some examples, the benchmark data set of variant calls includes an image-based benchmark data set of variant calls. The neural network model may include at least one of a vision transformer (ViT) or a convolutional neural network (CNN). Optionally, the neural network model may include one or more convolutional neural network (CNN) layers. In some implementations, training the neural network model includes training the neural network model using hyperparameter tuning.

Training the neural network model may further include training the neural network model on the pileup images to teach the neural network model to learn how to generate corresponding confidence scores for the variants identified in the output. In some examples, training the neural network model further includes training the neural network model on the pileup images to teach the neural network model to learn how to generate corresponding variant allele frequencies (VAFs) and/or corresponding VAF confidence intervals for the variants identified in the output. Optionally, the neural network model is trained on the pileup images to teach the neural network model to learn how to generate the output identifying variants that include single nucleotide variants (SNVs), insertions, or deletions. In some implementations, the neural network model is trained on the pileup images to teach the neural network model to learn how to generate the output identifying variants without imputation based on information from a reference panel.

In some examples, the operations may further include obtaining a test sequencing data set including a plurality of testing sequencing data inputs each obtained by sequencing a corresponding nucleic acid sequence from a corresponding test sample, and for each corresponding testing sequencing data input, a corresponding benchmark data set of variant calls for the corresponding nucleic acid sequence. In some additional examples, after training the neural network model, for each testing sequence data input among the plurality of testing sequencing data inputs of the sequencing data test set, the operations include processing, using the trained neural network model, the testing sequence data input to predict output variant calls identified in the corresponding nucleic acid sequence, and assessing, using one or more metrics, the predicted output variant calls based on the corresponding benchmark data set of variant calls for the corresponding nucleic acid sequence. In some implementations, the one or more metrics include at least one of accuracy, recall, or F-1 scores.

Another aspect of the disclosure provides a system that includes data processing hardware and memory hardware storing instructions that when executed on the data processing hardware causes the data processing hardware to perform operations for training a neural network model to learn how to generate output variant calls from sequencing data inputs obtained from sequencing nucleic acid sequences at depths of 10× or less from biological samples. The operations include receiving a plurality of sequencing data files collected from a population of subjects, each sequencing data file obtained from sequencing a nucleic acid sequence from a corresponding biological sample collected from a corresponding subject in the population of subjects. For each corresponding sequencing data file among the plurality of sequencing data files, the operations include receiving a corresponding set of variant call format (VCF) files generated for the corresponding sequencing data file, each respective VCF file in the corresponding set of VCF files generated as output from a respective one of a plurality of independent variant caller applications that receive the corresponding sequencing data file as input, each respective VCF file in the corresponding set of VCF files identifying variants. For each of one or more of the variants identified by each respective VCF file in the corresponding set of VCF files, the operations include generating a respective pileup image. The operations also include training the neural network model on the pileup images generated for the plurality of sequencing data files to teach the neural network model to learn how to generate an output identifying variants from sequencing data inputs obtained from sequencing nucleic acid sequences at depths of 10× or less from one or more other biological samples.

This aspect of the disclosure may include one or more of the following optional features. In some implementations, the corresponding sequencing data file includes a sequence alignment file (SAF) obtained from the sequencing of the nucleic acid sequence from the corresponding biological sample. In some examples, the nucleic acid sequence includes a deoxyribonucleic acid (DNA) sequence or a ribonucleic acid (RNA) sequence. Optionally, the nucleic acid sequence may include a deoxyribonucleic acid (DNA) sequence. The nucleic acid sequence may include a sequence of at least 50 nucleotides. In some implementations, the sequencing of the nucleic acid sequence from the corresponding biological sample includes paired end or single end sequencing. In some examples, the sequencing of the nucleic acid sequence from the corresponding biological sample includes whole genome sequencing or whole exome sequencing.

Optionally, the sequencing of the nucleic acid sequence from at least one of the plurality of biological samples is performed using whole genome sequencing and the sequencing of the nucleic acid sequence from at least another one of the plurality of biological samples is performed using whole exome sequencing. In some implementations, the sequencing of the nucleic acid sequence from the corresponding biological sample includes germline sequencing of the nucleic acid sequence from the corresponding biological sample. In these implementations, the germline sequencing may be performed at a depth of at least 50x. In some examples, the sequencing of the nucleic acid sequence from the corresponding biological sample includes somatic sequencing of the nucleic acid sequence from the corresponding biological sample. In these examples, the somatic sequencing may be performed at a depth of at least 150x.

The plurality of biological samples collected from the population of subjects may include a subset of biological samples collected from a respective subject in the population of subjects. In some implementations, for at least one corresponding biological sample from the subset of biological samples collected from the respective subject, the sequencing of the nucleic acid sequence from the at least one corresponding biological sample includes somatic sequencing of the nucleic acid sequence from the at least one corresponding biological sample from the subset of biological samples collected from the respective subject, and for at least another corresponding biological sample from the subset of biological samples collected from the respective subject, the sequencing of the nucleic acid sequence from the at least another corresponding biological sample includes germline sequencing of the nucleic acid sequence from the at least another corresponding biological sample from the subset of biological samples collected from the respective subject. Optionally, two or more biological samples in the subset of biological samples collected from the respective subject are collected at different timepoints. In some examples, the sequencing of the nucleic acid sequence from the two or more biological samples collected from the respective subject at different timepoints includes somatic sequencing of the nucleic acid sequence from the two or more biological samples. In some implementations, the two or more biological samples collected from the respective subject at different timepoints include tumor biopsy samples. The two or more biological samples collected from the respective subject at different timepoints may include liquid biopsy samples. In these examples, the liquid biopsy samples may include at least one of blood samples, urine samples, sputum samples, or cerebrospinal fluid samples.

In some implementations, the corresponding sequencing data file includes one or more quality control (QC) parameters for one or more loci in the nucleic acid sequence sequenced from the biological samples. Optionally, the corresponding sequencing data file may include one or more QC parameters for each locus in the nucleic acid sequence sequenced from the corresponding biological samples. In some examples, the operations further include, for the variants identified by the corresponding set of VCF files generated for the corresponding sequencing data file, filtering the variants based on the one or more QC parameters of the corresponding sequencing data file to remove any variants in the corresponding set of VCF files that are artifacts. Here, generating the respective pileup image for each of the one or more of the variants identified by each respective VCF file in the corresponding set of VCF files includes generating the respective pileup image for each of the one or more of the variants after filtering to remove the variants in the corresponding set of VCF files that are artifacts.

At least a portion of the biological samples collected from the population of subjects may include biological samples not preserved with a preservative or cross-linking agent. In some implementations, the at least the portion of the biological samples not preserved with the preservative or cross-linking agent include fresh biological samples. In some examples, the biological samples collected from the population of subjects include formalin-fixed paraffin-embedded (FFPE) samples. One or more data augmentation techniques may be performed on the corresponding sequencing data file received as input by the plurality of independent variant caller applications. Optionally, the variants identified by each respective VCF file in the corresponding set of VCF files may include at least one of single nucleotide variants (SNVs), insertions, or deletions.

In some implementations, the operations further include, for at least one corresponding sequencing data file, obtaining a corresponding benchmark data set of variant calls for the nucleic acid sequence sequenced from the corresponding biological sample, and for each respective VCF file in the corresponding set of VCF files generated for the corresponding sequencing data file, annotating, using the benchmark data set of variant calls, the respective pileup image generated for each of the one or more of the variants identified by the respective VCF file to flag any variants correctly identified by the respective VCF file, any variants incorrectly identified by the respective VCF file, and any variants unidentified by the respective VCF file. Here, training the neural network model on the pileup images includes training the neural network on the annotated pileup images. In some examples, the benchmark data set of variant calls includes an image-based benchmark data set of variant calls. The neural network model may include at least one of a vision transformer (ViT) or a convolutional neural network (CNN). Optionally, the neural network model may include one or more convolutional neural network (CNN) layers. In some implementations, training the neural network model includes training the neural network model using hyperparameter tuning.

Training the neural network model may further include training the neural network model on the pileup images to teach the neural network model to learn how to generate corresponding confidence scores for the variants identified in the output. In some examples, training the neural network model further includes training the neural network model on the pileup images to teach the neural network model to learn how to generate corresponding variant allele frequencies (VAFs) and/or corresponding VAF confidence intervals for the variants identified in the output. Optionally, the neural network model is trained on the pileup images to teach the neural network model to learn how to generate the output identifying variants that include single nucleotide variants (SNVs), insertions, or deletions. In some implementations, the neural network model is trained on the pileup images to teach the neural network model to learn how to generate the output identifying variants without imputation based on information from a reference panel.

In some examples, the operations may further include obtaining a test sequencing data set including a plurality of testing sequencing data inputs each obtained by sequencing a corresponding nucleic acid sequence from a corresponding test sample, and for each corresponding testing sequencing data input, a corresponding benchmark data set of variant calls for the corresponding nucleic acid sequence. In some additional examples, after training the neural network model, for each testing sequence data input among the plurality of testing sequencing data inputs of the sequencing data test set, the operations include processing, using the trained neural network model, the testing sequence data input to predict output variant calls identified in the corresponding nucleic acid sequence, and assessing, using one or more metrics, the predicted output variant calls based on the corresponding benchmark data set of variant calls for the corresponding nucleic acid sequence. In some implementations, the one or more metrics include at least one of accuracy, recall, or F-1 scores.

Another aspect of the disclosure provides a computer-implemented method executing on data processing hardware that causes the data processing hardware to perform operations for using a trained neural network model to identify variants in a nucleic acid sequence based on aligned sequencing reads obtained by sequencing the nucleic acid sequence at a depth of 10× or less. The operations include receiving aligned sequencing reads, wherein the sequencing reads are obtained by sequencing a nucleic acid sequence at a depth of 10× or less from a biological sample collected from a subject. The operations also include processing, using a trained neural network model, the aligned sequencing reads. Processing the aligned sequencing reads includes generating a respective pileup image for each locus in the nucleic acid sequence, and processing the pileup images generated for each locus in the nucleic acid sequence to identify variants and generate an output including an identification of variants in the nucleic acid sequence.

This aspect of the disclosure may include one or more of the following optional features. In some implementations, the output generated by processing the pileup images further includes at least one of corresponding confidence scores, corresponding VAFs, and/or corresponding VAF confidence intervals for the variants included in the identification of variants in the nucleic acid sequence. In some examples, the output includes a variant call format (VCF) file. Receiving the aligned sequencing reads may include receiving a sequencing alignment file. The sequence alignment file may optionally include a binary alignment map (BAM) file.

In some implementations, the trained neural network processes the pileup images to identify variants and generates the identification of variants without using imputation based on information from a reference panel. The sequencing reads may be obtained by sequencing the nucleic acid sequence at a depth of 4× or less. In some examples, the variants include germline variants. Optionally, the variants may include somatic variants. In some implementations, the variants include at least one of single nucleotide variants (SNVs), insertions, or deletions.

The operations may further include receiving subject data associated with the subject from which the biological sample was collected. Herem, processing the pileup images generated for each locus in the nucleic acid sequence further includes processing the subject data associated with the subject to generate the output. In some examples, the subject data associated with the subject includes electronic medical record (EMR) data associated with the subject. Optionally, the subject data associated with the subject may include sample data associated with the biological sample collected from the subject. In some implementations, the sample data associated with the biological sample includes at least one of: sample quality data for the biological sample; pathology data for the biological sample; biomarker data for the biological sample or for a separate biological sample collected from the subject; or epigenetic data for the biological sample.

The sequencing may include whole genome sequencing. In some examples, the sequencing includes whole exome sequencing. The trained neural network model may optionally include at least one of a vision transformer (ViT) or a convolutional neural network (CNN). In some implementations, the trained neural network model includes one or more convolutional neural network (CNN) layers.

In some examples, the trained neural network model includes a convolutional neural network (CNN) multi-module architecture. The CNN multi-module architecture may include a pileup visualization layer configured to receive, as input, aligned sequencing reads obtained by sequencing the nucleic acid sequence at the depth of 10× or less from the biological sample collected from the subject and generate, as output, the respective pileup image for each locus in the nucleic acid sequence. The CNN multi-module architecture may also include a first CNN layer configured to receive, as input from the pileup visualization layer, the respective pileup image generated for each locus in the nucleic acid sequence; and generate, as output, the output including the identification of variants in the nucleic acid sequence. In some implementations, the output generated as output from the first CNN layer further includes at least one of the corresponding confidence scores, the corresponding VAFs, and/or the corresponding VAF confidence intervals for the variants included in the identification of variants in the nucleic acid sequence.

The CNN multi-module architecture may further include a second CNN layer configured to receive, as input, subject data associated with the subject and at least one of the output of the first CNN layer or the respective pileup image for each locus in the nucleic acid sequence generated as output from the pileup visualization layer; and generate, as output, a refined output including a refined identification of variants in the nucleic acid sequence. In some examples, the refined output generated as output from the second CNN layer further includes at least one of corresponding confidence scores, corresponding VAFs, and/or the corresponding VAF confidence intervals for the variants included in the refined identification of variants in the nucleic acid sequence. Optionally, the subject data associated with the subject may include electronic medical record (EMR) data associated with the subject. In some implementations, the subject data associated with the subject includes sample data associated with the biological sample collected from the subject. The sample data associated with the biological sample may include at least one of: sample quality data for the biological sample; pathology data for the biological sample; biomarker data for the biological sample or for a separate biological sample collected from the subject; or epigenetic data for the biological sample.

Another aspect of the present disclosure provides a system that includes data processing hardware and memory hardware in communication with the data processing hardware and storing instructions that when executed on the data processing hardware causes the data processing hardware to perform operations for using a trained neural network model to identify variants in a nucleic acid sequence based on aligned sequencing reads obtained by sequencing the nucleic acid sequence at a depth of 10× or less. The operations include receiving aligned sequencing reads, wherein the sequencing reads are obtained by sequencing a nucleic acid sequence at a depth of 10× or less from a biological sample collected from a subject. The operations also include processing, using a trained neural network model, the aligned sequencing reads. Processing the aligned sequencing reads includes generating a respective pileup image for each locus in the nucleic acid sequence, and processing the pileup images generated for each locus in the nucleic acid sequence to identify variants and generate an output including an identification of variants in the nucleic acid sequence.

This aspect of the disclosure may include one or more of the following optional features. In some implementations, the output generated by processing the pileup images further includes at least one of corresponding confidence scores, corresponding VAFs, and/or corresponding VAF confidence intervals for the variants included in the identification of variants in the nucleic acid sequence. In some examples, the output includes a variant call format (VCF) file. Receiving the aligned sequencing reads may include receiving a sequencing alignment file. The sequence alignment file may optionally include a binary alignment map (BAM) file.

In some implementations, the trained neural network processes the pileup images to identify variants and generates the identification of variants without using imputation based on information from a reference panel. The sequencing reads may be obtained by sequencing the nucleic acid sequence at a depth of 4× or less. In some examples, the variants include germline variants. Optionally, the variants may include somatic variants. In some implementations, the variants include at least one of single nucleotide variants (SNVs), insertions, or deletions.

The operations may further include receiving subject data associated with the subject from which the biological sample was collected. Herem, processing the pileup images generated for each locus in the nucleic acid sequence further includes processing the subject data associated with the subject to generate the output. In some examples, the subject data associated with the subject includes electronic medical record (EMR) data associated with the subject. Optionally, the subject data associated with the subject may include sample data associated with the biological sample collected from the subject. In some implementations, the sample data associated with the biological sample includes at least one of: sample quality data for the biological sample; pathology data for the biological sample; biomarker data for the biological sample or for a separate biological sample collected from the subject; or epigenetic data for the biological sample.

The sequencing may include whole genome sequencing. In some examples, the sequencing includes whole exome sequencing. The trained neural network model may optionally include at least one of a vision transformer (ViT) or a convolutional neural network (CNN). In some implementations, the trained neural network model includes one or more convolutional neural network (CNN) layers.

In some examples, the trained neural network model includes a convolutional neural network (CNN) multi-module architecture. The CNN multi-module architecture may include a pileup visualization layer configured to receive, as input, aligned sequencing reads obtained by sequencing the nucleic acid sequence at the depth of 10x or less from the biological sample collected from the subject and generate, as output, the respective pileup image for each locus in the nucleic acid sequence. The CNN multi-module architecture may also include a first CNN layer configured to receive, as input from the pileup visualization layer, the respective pileup image generated for each locus in the nucleic acid sequence; and generate, as output, the output including the identification of variants in the nucleic acid sequence. In some implementations, the output generated as output from the first CNN layer further includes at least one of the corresponding confidence scores, the corresponding VAFs, and/or the corresponding VAF confidence intervals for the variants included in the identification of variants in the nucleic acid sequence.

The CNN multi-module architecture may further include a second CNN layer configured to receive, as input, subject data associated with the subject and at least one of the output of the first CNN layer or the respective pileup image for each locus in the nucleic acid sequence generated as output from the pileup visualization layer; and generate, as output, a refined output including a refined identification of variants in the nucleic acid sequence. In some examples, the refined output generated as output from the second CNN layer further includes at least one of corresponding confidence scores, corresponding VAFs, and/or the corresponding VAF confidence intervals for the variants included in the refined identification of variants in the nucleic acid sequence. Optionally, the subject data associated with the subject may include electronic medical record (EMR) data associated with the subject. In some implementations, the subject data associated with the subject includes sample data associated with the biological sample collected from the subject. The sample data associated with the biological sample may include at least one of: sample quality data for the biological sample; pathology data for the biological sample; biomarker data for the biological sample or for a separate biological sample collected from the subject; or epigenetic data for the biological sample.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1 is a schematic view of an example system for training and using a sequencing variant caller model.

FIGS. 2A and 2B are schematic views of example architectures of the sequencing variant caller model of FIG. 1.

FIG. 5 is a schematic view of an example computing device that may be used to implement the systems and methods described herein.

Like reference symbols in the various drawings indicate like elements.

### DETAILED DESCRIPTION

### Definitions

As used herein, the “depth” of sequencing refers to the average number of times each nucleotide in a nucleic acid sequence is determined or read using a sequencing process.

As used herein, the terms “variant call(s)” and “variant calling” refer to detecting a variant or mutation at a particular locus in a nucleic acid sequence, e.g., a DNA or RNA sequence. For instance, a variant may include a single nucleotide variant (SNV), insertion, deletion or other variation. Variants that may be identified using the techniques disclosed herein include nucleotide variants such as germline variants or somatic variants. Germline variants occur in all cells of an individual, including germ cells (sperm or egg cells). They are inherited variants that are present throughout an individual's lifetime and are passed to offspring. One type of germline variant is a single nucleotide polymorphism (SNP), which is a type of SNV that occurs at a frequency of more than 1% of the population. In some implementations, the variant caller disclosed herein is trained to detect germline variants. In contrast to germline variants, somatic variants are acquired during an individual's lifetime, occur in non-reproductive or somatic cells, and are not inherited by offspring. A somatic variant affects a cell (for instance, a tumor cell) and its progeny. An accumulation of mutations in DNA may cause cancer. A cancerous tumor can be heterogeneous, including some normal cells, some cells with few mutations indicating an early stage of cancer progression, and some cells with more mutations indicating a later-stage cancer progression. When sequencing nucleic acid sequences from a tumor tissue, the heterogeneous characteristic of the tumor tissue can result in somatic mutations appearing at a low frequency. In some implementations, the variant caller disclosed herein is trained to detect somatic variants.

As used herein, sequencing of a nucleic acid sequence at depth coverages of 10× or less may be referred to as “low pass” (LP) sequencing.

The terms “position” and “locus” refer to a location or coordinate of one or more nucleotides within a nucleic acid sequence. The terms “position” and “locus” can also refer to a location or coordinate of one or more base pairs in nucleic acid sequence.

Implementations herein are directed toward training and using an artificial intelligence (AI)-based variant caller to improve accuracy and confidence of variant calling, particularly for low frequency variants. More specifically, the AI-based variant caller and related methods disclosed herein allow for variant calling in nucleic acid sequences sequenced at low depths, such as depths of 10× or less. The ability to use low depth sequencing data allows data to be obtained at lower cost, and it is also less computationally intensive to process, thereby providing both cost and computational improvements in the technological field of variant calling. Advantageously, the AI-based variant caller and methods disclosed herein make it possible to call variants in nucleic acid sequences sequenced at low depths without the need for imputation using information from a reference panel. By eliminating the need to perform imputation, the variant calls output by the AI-based variant caller advantageously avoid inaccuracies that can result from the use of a reference panel for imputation. Additionally, analysis of somatic-derived variants would not be evaluable using imputation given the reference panel leveraged for imputations is intended for germline genotyping. Furthermore, the AI-based variant caller and methods disclosed herein provide other advantages, such as providing enhanced sensitivity for detecting subtle changes in variant allele frequencies, which is important for early detection of diseases such as cancer, and improving variant calling accuracy from nucleic acid sequences sequenced from lower quality biological samples and/or biological samples with smaller masses of DNA.

Next generation sequencing (NGS) technologies such as whole genome sequencing (WGS) and whole exome sequencing (WES) are technologies that enable concurrent analysis of thousands to millions of genetic fragments. These massively parallel sequencing technologies can be valuable for identifying genetic variants from patient samples in clinical settings. However, conducting WGS and WES at standard depth coverages (e.g., 30× or greater) that are needed to confidently call target genomic variants has a relatively high cost. Also, it is operationally challenging to deploy these standard depth coverage sequencing methods for target genomic studies because of the limited availability of suitable biological samples (e.g., samples with a sufficient mass of DNA) and the relatively high cost and computational time requirements needed to analyze complex genomic datasets.

Often the depth coverage of WGS and WES ranges from 50× (for normal mean coverages) up to around 150× (for confidently calling target genomic variants at the somatic level). WGS or WES performed at standard coverage depths can have a high cost for each biological sample for which the sequencing is performed. By contrast, sequencing at lower coverage depths can be performed at a substantially lower cost.

Additionally, most current genomic profiling protocols require biological samples to have relatively large DNA input masses (e.g., 100-400 ng of DNA), usually in conjunction with polymerase chain reaction (PCR)-free library prep chemistries. Isolating this amount of DNA from low quality formalin-fixed, paraffin-embedded (FFPE) tissue samples can be challenging, and low quality samples may be the only samples that are available. Further complexity is introduced when analyzing somatic variants, because sequencing needs to be performed on a normal sample to filter out any germline-derived variants from the downstream tumor genomic profiling.

Through the technique of genotyping array, costs of identifying genetic variants from the DNA of biological samples can be reduced by performing WGS or WES at lower depth coverages (10× or less, e.g., ~0.1×-5×). Genotyping array allows for identification of nucleotides from some fixed points in the DNA sequence, whereby the rest of the DNA sequence may be imputed. For the imputation, a reference panel including DNA sequences collected from a population of individuals is used to fill in the gaps in the DNA sequence. As such, genotyping imputation techniques that leverage reference panels can, in some instances, accurately identify common variants in the germline DNA sequence. However, the likelihood of accurately identifying variants is reduced in some circumstances, such as when (i) the variants are less common or rare, and/or (ii) the biological sample that is being assessed has been obtained from an individual of different ancestry than the population from which samples were obtained to make the reference panel used for the genotype imputation.

Implementations herein are directed toward training a neural network model on pileup images generated for a plurality of sequencing data files to teach the neural network model to learn how to generate output variant calls from sequencing data inputs obtained from sequencing nucleic acid sequences at depths of 10× or less from biological samples. Initially, techniques disclosed herein include receiving the plurality of sequence data files collected from a population of subjects, wherein each sequencing data file is obtained from sequencing a nucleic acid sequence from a corresponding biological sample collected from a corresponding subject in the population of subjects. The sequencing data files may each include or consist of a sequence alignment file such as, e.g., a binary alignment map (BAM) file, that stores the precise genomic locations or positions where each sequencing read in the nucleic acid sequence aligns to a reference genome. Thereafter, each corresponding sequencing data file among the plurality of sequencing data files is provided as input to each of a plurality of different variant caller applications to generate a corresponding set of variant call format (VCF) files. Here, each VCF file in the corresponding set of VCF files is generated as output from a respective one of the plurality of different variant caller applications and identifies variants in the nucleic acid sequence. The pileup images used for training the neural network model are obtained by generating a respective pileup image for each of one or more of the variants identified by each respective VCF file in the corresponding set of VCF files generated for each of the plurality of sequencing data files. Accordingly, the plurality of variant caller applications are leveraged during training to generate the VCF files from a corresponding BAM and the pileup images ultimately used for training the model are generated based on the variants identified in the VCF files.

While the resulting trained neural network model is capable of generating output variant calls based on sequencing nucleic acid sequences at depths of 10× or less from biological samples, the sequencing of the nucleic acid sequence from the corresponding biological sample associated with each corresponding sequencing data file obtained for training the neural network model is typically performed at depths of at least 50× when the sequencing includes germline sequencing or at depths of at least 150× when the sequencing includes somatic sequencing. Notably, the neural network model is trained to learn how to generate the output variant calls from sequencing data inputs (e.g., BAM files) obtained from sequencing nucleic acid sequences at depths of 10× or less without performing any imputation based on information from a reference panel.

Implementations herein are further directed toward using a trained neural network model to identify variants in a nucleic acid sequence based on aligned sequencing reads (e.g., a sequencing alignment file, such as a BAM file) obtained by sequencing the nucleic acid sequence at a depth of 10× or less from a biological sample collected from a subject. Here, the sequencing alignment file is input to the trained neural network model and the trained neural network model processes the sequencing alignment file by generating a respective pileup image for each locus (or for a subset of loci) in the nucleic acid sequence, and processing the pileup images to generate, as output, an identification of variants in the nucleic acid sequence and, optionally: corresponding confidence scores; corresponding variant allele frequencies (VAFs); and/or corresponding VAF confidence intervals for the variants. In some implementations, the output is in the form of a variant call format (VCF) file.

In some implementations, the trained neural network model additionally processes subject data associated with the subject from which the biological sample was collected. In some implementations, processing the subject data serves the purpose of providing refined output. In these implementations, the output may include a refined identification of variants (e.g., so as to identify the variants with improved accuracy) in the nucleic acid sequence. Additionally or alternatively, the output may include refined confidence scores, refined VAFs, and/or refined VAF confidence intervals for the corresponding identified variants. For instance, the trained neural network model may process the pileup images and the subject data associated with the subject to generate, as output, the VCF file providing the refined identification of variant calls and optionally a corresponding confidence score for each variant in the refined identification of variants. Here, each confidence score reflects an overall confidence or likelihood that a corresponding variant call (a variant as identified by the output) is accurate. In other words, the subject data may act as weights to enable the trained neural network model to output confidence scores for the variant calls revealed in the pileup images. The subject data associated with the subject may include electronic medical record (EMR) data associated with the subject. Additionally or alternatively, the subject data associated with the subject may include sample data associated with the biological sample collected from the subject. Here, the sample data associated with the biological sample may include at least one of sample quality data for the biological sample, pathology data for the biological sample, biomarker data for the biological sample or for a separate biological sample collected from the subject, or epigenetic data (such as, for instance, the methylation profile of the nucleic acid sequence) for the biological sample.

As will become apparent, the trained neural network provides a refined identification of variant calls with high confidence scores, which can be used for diagnosing, prognosticating, and/or monitoring a disease or condition of the subject. For instance, the disease may include a cancer, an autoimmune disease, a cardiovascular disease, or a neurologic disease. Determining nucleic acid variants, e.g., somatic variants such as DNA mutations that have occurred in a tumor, can have dramatic impact in many clinical settings. For example, determining nucleic acid variants can improve diagnosis, treatment, and management of a disease or treatment of the subject. For example, the variant profile as determined using technologies described herein can allow precision medicine approaches to improve treatment (which may be a preventative treatment) for a disease or condition of the subject. For instance, it can enable selection of a targeted therapy and/or earlier detection of resistance to treatment.

One way to find out what variants are present in a diseased tissue of a person is to take out the genetic material from the diseased tissue and compare it with the genetic material from a normal tissue (e.g., healthy tissue) of the same person. The comparison can be done using sequencing of the genetic material, for example, sequencing reads from NGS. Here, the sequencing read performed on the normal tissue may include germline variants that are hereditary. As such, to identify somatic variants unique to the diseased tissue, the frequency of variants identified in the normal tissue can be subtracted from the frequency of variants identified in the diseased tissue. Notably, the sequencing costs to perform these comparisons is compounded because at least two separate NGS reads are required, i.e., at least one NGS performed on the diseased tissue and at least one NGS performed on the healthy tissue.

The task of identifying a variant at a certain location in a nucleic acid sequence (e.g., a DNA or RNA sequence) as described herein can be seen as a sorting job and referred to as “variant calling”. While many tools may use different rules with very different results, the methods, systems, and devices of the present disclosure use the trained neural network for variant calling, and more specifically, apply the trained neural network to a sequence alignment file (e.g., BAM file) to match the sequencing read of a nucleic acid sequence from a biological sample (e.g., tissue) to a reference genome to distinguish between a nucleotide variant that is in the real DNA sequence from a false nucleotide variant that is erroneously identified in the sequencing reads.

A germline sample is a sample of DNA that is inherited from one's parents and is present in all the cells of the body, except for the sperm or egg cells. A germline sample can be obtained from various sources, such as blood, saliva, hair, or cheek swabs. A germline sample can be used to study the genetic variation and ancestry of an individual, as well as to identify inherited diseases or predispositions. A germline sample can also be compared to a somatic sample, which is a sample of DNA that is acquired from a specific tissue or organ and may have mutations or alterations that are not present in the germline. A somatic sample can be used to diagnose or monitor cancers or other diseases that affect specific cells or tissues.

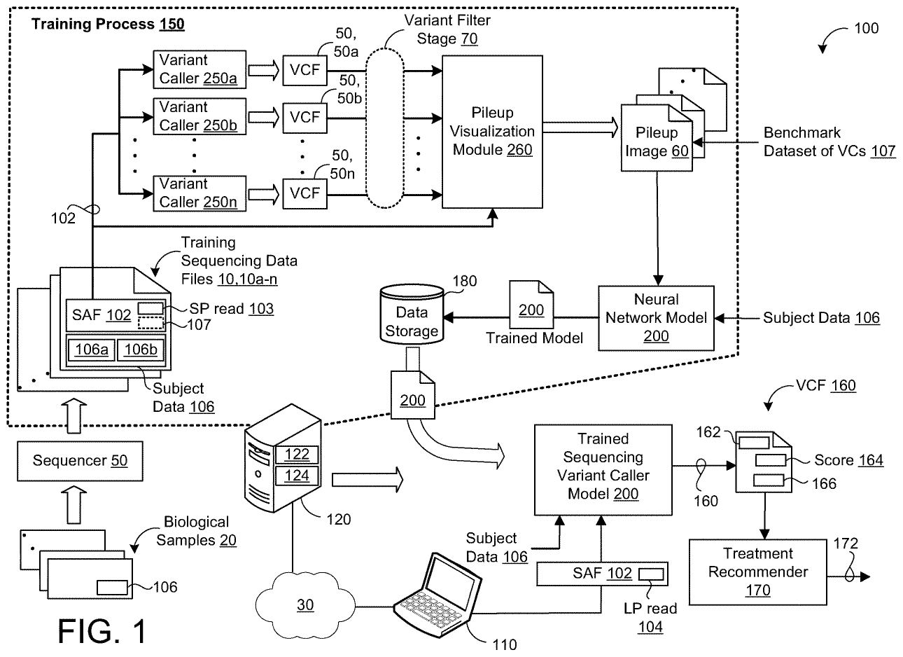

FIG. 1 shows an example system 100 for training a neural network model 200 to provide a trained sequencing variant caller model 200 (interchangeable referred to as “trained neural network model” or “trained model”) for use in detecting nucleotide variants, e.g., from a low-pass (LP) sequencing read 104 performed on a nucleic acid sequence from a biological sample 20, without performing imputation based on information from a reference panel. The LP sequencing read 104 corresponds to sequencing data from sequencing of the nucleic acid sequence at a depth coverage of 10× or less. For instance, the LP sequencing read 104 may include a next generation sequencing (NGS) read of nucleic acids at lower depth coverages (~0.1-5×) or moderate depth coverages (~5-10×). Specifically, the trained sequencing variant caller model 200 receives, as input, a sequencing alignment file (e.g., a binary alignment map (BAM) file) 102 that stores the precise genomic location or position where each nucleotide identified in the nucleic acid sequence (i.e., the LP sequencing read 104) aligns to a reference genome, and generates, as output, an output 160 including an identification of variants 162 in the nucleic acid sequence from the biological sample 20 collected from a subject. The output 160 may be in the form of one or more files, e.g., one or more variant call format files (VCF) files 160. In some implementations, the output 160 includes metadata 167 including corresponding variant allele frequency (VAF) 166 and/or VAF confidence intervals 166 for variants included in the identification of variants 162. In some examples, the output 160 includes confidence scores 164 for variants included in the identification of variants 162.

The system 100 includes a computing system 120 having data processing hardware 122 and memory hardware 124 in communication with the data processing hardware 122 and storing instructions that cause the data processing hardware 122 to perform operations. The computing system 120 may store the trained neural network model 200 in data storage 180. In some implementations, the computing system 120 (e.g., the data processing hardware 122) provides the trained neural network model 200 to a user computing device 110 via a network 30, or the user computing device 110 accesses the trained neural network model 200 from the data storage 180. In some examples, the user computing device 110 receives a sequencing alignment file, such as a SAF (e.g., BAM file) 102, obtained by sequencing a nucleic acid sequence at a depth of 10× or less from a biological sample collected from a subject and executes the trained neural network model 200 to process the SAF (e.g., BAM file) 102 to generate a VCF file 160 that provides identification of variant calls 162 in the nucleic acid sequence. Sequencing data obtained from sequencing a nucleic acid sequence at the depth of 10x or less corresponds to a LP sequencing read 104. For instance, the LP sequencing read 104 may include sequencing data obtained from next generation sequencing (NGS) at lower depth coverages (~0.1-5×) or moderate depth coverages (~5-10×).

While the resulting trained neural network model 200 is capable of generating output variant calls 162 based on LP sequencing reads 104 of nucleic acid sequences from biological samples, a training process 150 trains the neural network model 200 based on standard-pass (SP) sequencing reads 103 of nucleic acid sequences from biological samples 20 collected from a population of subjects. The SP sequencing reads 103 correspond to nucleic acid sequences sequenced at standard depth coverages, e.g., least 30×. In some implementations, the SP sequencing is at coverages of at least 50× for sequences obtained by germline sequencing or at depth coverages of at least 150× for sequences obtained by somatic sequencing. In some implementations, SP sequencing reads 103 include nucleic acid sequences sequenced at depth coverages of at least 50×, 100×, or 150×. The SP sequencing reads 103 may include sequencing data obtained from Whole Genome Sequencing (WGS) or Whole Exome Sequencing (WES).

The biological samples 20 from which nucleic acids are sequenced to obtain input sequencing data (SP read 103) for use by the training process 150, or to obtain sequencing data 10 that are used to provide sequence alignment files 102 to the trained model 200, can include a variety of types of biological samples, such as, for instance blood samples (e.g., whole blood samples), buccal swab samples, saliva samples, mouthwash samples, liquid biopsy samples, skin biopsy samples, cultured cell samples, bone marrow samples, hair samples, cerebrospinal fluid samples, urine samples, or other tissue samples (e.g., tumor samples). The biological samples 20 may be or may include biological samples not preserved with a preservative or cross-linking agent. For instance, the biological samples may be or may include fresh biological samples. In some instances, the biological samples may be or may include formalin-fixed paraffin-embedded (FFPE) samples.

The training process 150 generates training data by initially receiving a plurality of sequencing alignments, which may be in the form of sequencing alignment files (such as, for instance, binary alignment map (BAM) files) 102 which are generated by aligning sequences from sequencing data files 10, 10a-n collected from the population of subjects. Each corresponding sequencing data file 10 is obtained from a sequencing of a nucleic acid sequence from a corresponding biological sample 20 collected from a corresponding subject in the population of subjects. The sequences used for training typically include multiple sequence reads obtained by standard pass (SP) sequencing 103. The nucleic acid sequences associated with each sequencing data file 10 may include a deoxyribonucleic acid (DNA) sequence or a ribonucleic acid (RNA) sequence. The nucleic acid sequence may include a sequence of at least 50 nucleotides, at least 100 nucleotides, or at least 150 nucleotides. Additionally, sequencing of the nucleic acid sequences may include paired end or single end sequencing. In some examples, the SP sequencing reads 103 from at least one of the plurality of biological samples 20 is performed using WGS and the SP sequencing reads from at least another one of the plurality of biological samples 20 is performed using WES.

Each sequence from SP sequencing reads 103 obtained from sequencing of a nucleic acid sequence from a biological sample 20 can be aligned to provide a sequence alignment file (SAF) (which may be, for instance, a binary alignment map (BAM) file) 102. That is, the SAF (e.g., BAM file) 102 associated with each biological sample stores the precise genomic locations or positions where each nucleic acid that is identified in the nucleic acid sequence with each SP sequencing read 103 aligns to a reference genome.

While examples herein depict each sequencing data file 10 including a BAM file 102, the sequencing data file 10 may be or include one or more other types of SAFs without departing from the scope of the present disclosure. In some implementations, raw sequencing data obtained from sequencing of a nucleic acid in a biological sample, and as included in sequencing data files 10 (which may be, e.g., FASTA files), are aligned with a reference genome to obtain a SAF (e.g., BAM file) 102 for the relevant biological sample.

In some implementations, a subset of at least two biological samples 20 from the plurality of biological samples 20 are collected from a respective subject in the population of subjects. In these implementations, SP sequencing reads 103 from at least one corresponding biological sample 20 in the subset of biological samples 20 collected from the respective subject may include somatic sequencing of the nucleic acid sequence from the at least one corresponding biological sample 20, while corresponding SP sequencing reads 103 from at least one other corresponding biological sample 20 in the subset of biological samples 20 collected from the respective subject may include germline sequencing of the nucleic acid sequence from the at least one other corresponding biological sample 20. For instance, in sequencing data files associated with subjects diagnosed with or at risk for disease (e.g., cancer), one biological sample 20 sequenced by germline sequencing may include a healthy/normal biological sample collected from the subject while another biological sample 20 sequenced by somatic sequencing may include a somatic biological sample (e.g., tumor tissue) collected from the same subject.

In some examples, each biological sample 20 in a subset of biological samples 20 is collected from a respective subject at a different timepoint than one or more other biological samples 20 (e.g., each other biological sample) in the subset of biological samples 20 collected from the respective subject. In these examples, the SP sequencing reads 103 for the subset of biological samples 20 collected from the respective subject may include somatic sequencing of the nucleic acid sequence from each biological sample 20 in the subset of biological samples 20 collected from the respective subject at the different timepoints. For instance, the subset of biological samples collected from the respective subject may include tumor biopsy samples. In this way, the tumor biopsy samples collected from the respective subject at different timepoints allow for monitoring disease progression using the trained sequencing variant caller model 200 for detecting the presence or absence of somatic variants at the different timepoints. In some scenarios, one or more of the biological samples collected from the respective subject may include liquid biopsy samples such as, without limitation, blood samples, urine samples, sputum samples, or cerebrospinal fluid samples.

Subject data 106 associated with the subject from which a corresponding biological sample 20 was collected may also be used by the training process 150 for training the neural network model 200 or in using the trained model 200 to identify variants. The subject data 106 may include electronic medical record (EMR) data 106a associated with the subject that may include demographic information, disease diagnosis/prognosis, a clinical trial name in which the subject participates, a medication list, an indication of the subject's responsiveness or failure to respond to a medication, family history, or other attributes associated with the subject that may be useful, e.g., for applications disclosed herein. Additionally or alternatively, the subject data 106 may include sample data 106b associated with the biological sample 20 collected form the subject such as, without limitation, sample quality data for the biological sample, pathology data for the biological sample, biomarker data for the biological sample or for a separate biological sample collected from the subject, and/or epigenic data for the biological sample. For instance, when the biological sample 20 is a tumor tissue sample, the sample data 106b may include pathology data represented by quality control (QC) parameters such as total tissue area of the tumor tissue sample, a percentage of necrosis, percentage of tissue, or any other parameter associated with the tumor tissue sample that may be useful for applications disclosed herein. Sample data 106b can include, e.g., epigenic data, such as methylation profiling data, which may be used by the training process 150 in training the neural network model 200 and/or in using the trained model 200. Including subject data 106 as input data for training and/or as input data for use of the trained model 200 can allow the model 200 to provide improved output 160. In some implementations, the improved output 160 incudes refined identification (e.g., improved accuracy of identification) of variants 162, or VAFs 166 or VAF intervals 166, or improved confidence scores 164 in the variants 162 or VAFs 166. As will become apparent, subject data 106 associated with a corresponding sequencing data file 10 may be used in the training process 150 for teaching the neural network model 200 to learn how to draw inferences between the variants identified by the independent variant caller applications and the subject data 106.

In some examples, one or more data augmentation techniques are performed on the sequencing data included in sequencing data file 10 used to create the sequence alignment file SAF (e.g., BAM file) 102 that is input to the plurality of variant caller applications 250. For instance, the sequencing data file 10 may be augmented using transformations or perturbations to increase the size and diversity of the sequencing data using AI-based or statistical techniques. Data augmentation may advantageously reduce overfitting the neural network model 200 to the data.

With continued reference to FIG. 1, for each corresponding SAF (e.g., BAM file) 102, the training process 150 receives a corresponding set of variant call format (VCF) files 50, 50a-n generated for the corresponding SAF 102 included in the corresponding sequencing data file 10. Here, each respective VCF file 50 in the corresponding set of VCF files 50 is generated as output from a respective one of a plurality of independent variant caller applications 250, 250a-n that receives the corresponding SAF (e.g., BAM file) 102 as input. Each respective VCF file 50 includes variants identified in the corresponding SAF (e.g., BAM file) 102 by the respective independent variant caller application 250. Notably, the independent variant caller applications 250 are only leveraged by the training process 150 for training the neural network model 200 and are not used by the trained neural network model 200 during inference when generating output variant calls. The variants identified by each respective VCF file 50 in the corresponding set of VCF files may include at least one of single nucleotide variants (SNVs), insertions, or deletions. In some examples, one or more of the VCF files 50 in the corresponding set of VCF files 50 indicate a variant allele frequency (VAF) and/or VAF confidence interval. The VAF represents a proportion of sequencing reads that support an alternate (variant) allele at a specific genomic location in the SAF (e.g., BAM file) 102. In simpler terms, the VAF indicates a percentage of times particular variant is identified at specific genomic location compared to a total number of reads at the specific genomic location. The VAF confidence interval represents a range of plausible values for a true VAF of a particular variant identified at a specific genomic location to account for uncertainty associated with estimating the VAF from a finite number of sequencing reads performed at the specific genomic location.

Described in greater detail below, the neural network model 200 includes at least one of a vision transformer (ViT) or a convolutional neural network (CNN) which are unable to interpret and process VCF files 50. For each of one the one or more variants identified by each respective VCF file 50 in the corresponding set of VCF files 50 generated from the same SAF (e.g., BAM file) 102, a pileup visualization module 260 generates, using the SAF 102, a respective pileup image 60. Each respective pileup image 60, or sometimes referred to as a “pileup visualization”, includes a graphical representation of aligned sequencing reads at a specific genomic location, thereby providing a visual-based stack of individual sequencing reads on top of each other at the specific genomic location. The pileup image 60 enables the neural network model (e.g., ViT or CNN) to learn to recognize patterns associated with variants. For instance, the pileup image 60 may provide a visualization of any combination of the following: a reference base; sequencing reads aligned to the position of the reference base; any variations from the reference base within those reads; any variants identified by the respective mutation callers; and the depth of coverage. By contrast, while the VCF files 50 provide information about the identified variants, such as SNVs, insertions, or deletions, as well as details regarding the position of each variant identified to provide a list of where differences exist between SP sequencing read 103 and the reference genome, the VCF files 50 are delimited text files that only provide a single variant per line that cannot be interpreted by the ViT and/or the CNN of the neural network model 200.

In some implementations, the subject data 106 includes sample data 106b providing one or more QC parameters, e.g., QC parameters for one or more loci in the SP sequencing read 103. In some examples, for the variants identified by the corresponding set of VCF files 50 generated for the corresponding sequencing data file 10, the training process 150 employs a filtering stage 70 to filter the variants based on the one or more QC parameters so as to remove any variants in the corresponding set of VCF files 50 that are artifacts (this filtering serves to remove variants that were incorrectly identified as variants). In these examples, the pileup visualization module 260 generates the respective pileup image for each of the one or more variants after such filtering.

Still referring to FIG. 1, after the visualization module 260 generates the pileup images 60, the training process 150 trains the neural network model 200 on the pileup images 60 generated for the plurality of sequencing data files 102 to teach the neural network model 200 to learn how to identify variants 162 from sequencing data inputs (i.e., LP sequence reads 104) obtained from sequencing nucleic acid sequences at depths of 10× or less from one or more other biological samples. The variants identified may include SNVs, insertions, or deletions. In some examples, the training process 150 further trains the neural network model 200 on the pileup images 60 to teach the neural network model to learn how to generate corresponding confidence scores 164 for the variants 162 identified in the output 160. In some additional examples, the training process 150 further trains the neural network model 200 on the pileup images to teach the neural network model to learn how to generate corresponding VAFs 166 and/or corresponding VAF confidence intervals 166 for the variants 162. The training process 150 may train the neural network model 200 on the pileup images 60 to teach the neural network model 200 to learn how to generate an output 160 (which can be, e.g., a VCF file) that includes the variants 162, the corresponding confidence scores 164, the corresponding VAFs 166, and/or the corresponding VAF confidence intervals 166.

Notably, the training process 150 trains the neural network model 200 to learn how to generate the output variant calls without requiring any imputation to be performed on information from a reference panel. The training process 150 may include using hyperparameter tuning to train the neural network model 200.

In some examples, the training process 150 trains the neural network model 200 on both the pileup images 60 and the corresponding subject data 106 associated with the subject from which the biological sample 20. In these implementations, the neural network model 200 uses the subject data 106 associated with the subject from which the biological sample 20 to further refine the information in the VCF file 160 which provides the output variant calls 162, and optionally also corresponding confidence scores 164, VAFs 166, and/or VAF confidence intervals 166. Here, the subject data 106 may act as weights applied in the neural network model to refine its output; for instance, the weights may serve to improve the accuracy of the variant calls and/or the improve corresponding confidence scores for the variant calls.

In some examples, at least one corresponding sequencing data file 10 or SAF (e.g., BAM file) 102 includes a corresponding benchmark dataset of variant calls (VCs) 107 for the SP sequencing read 103 performed on the corresponding biological sample 20. The benchmark dataset of VCs 107 may include ground-truth labels of variants that differ from the reference genome against which the SP sequencing reads 103 are aligned in the SAF (e.g., BAM file) 102. These ground-truth labels of variants can be diluted in levels needed to verify that the independent variant caller applications 250 are accurately identifying the variants. For each respective VCF file 50 in the corresponding set of VCF files 50 generated for the corresponding sequencing alignment file 102, the training process 150 may annotate, using the benchmark dataset of VCs 107, the respective pileup image 60 generated for each of the one or more variants identified by the respective VCF file 50 to flag: any variants correctly identified by the respective VCF file 50; any variants incorrectly identified by the respective VCF file 50; and any variants unidentified by the respective VCF file 50. Here, training the neural network model 200 on the pileup images 60 may include training the neural network model 200 on the annotated pileup images 60. The benchmark dataset of VCs 107 may include an image-based benchmark dataset of variant calls. The benchmark dataset of VCs 107 may include a Genome In A Bottle (GIAB) dataset providing ground-truth labels for germline variants. The benchmark dataset of VCs 107 may additionally or alternatively include digital PCR ground-truth labels for somatic variants.

In some implementations, after the neural network model 200 is trained by the training process 200, the trained neural network model 200 is tested/evaluated on a test sequencing data set that includes a plurality of testing sequencing data inputs each obtained by sequencing a corresponding nucleic acid sequence from a corresponding test sample and a corresponding benchmark dataset of variant calls for each corresponding nucleic acid sequence. Each test sequencing data input may include a corresponding sequence alignment file (e.g., BAM file) that stores the precise genomic locations or positions where each sequencing read in the nucleic acid sequence aligns to a reference genome. The benchmark data set of variant calls 107 may include ground-truth labels of variants that differ from the reference genome to which the nucleic acid sequence is aligned in the sequence alignment file (e.g., BAM file) 102. In some examples, the sequencing of the nucleic acid sequences is performed at depths less than 10×. For each testing sequence data input, the trained neural network model 200 may process the testing sequence data input (e.g., BAM) to predict output variant calls identified in the nucleic acid sequence and one or more metrics may be used to assess the predicted output variant calls based on the corresponding benchmark dataset of variant calls for the corresponding nucleic acid sequence. The one or more metrics may include at least one of accuracy, recall, or F-1 scores.

After the sequencing variant caller model 200 is trained by the training process 150 and optionally validated/tested, the trained sequencing variant caller model 200 can be used to identify variants in aligned sequencing reads of a nucleic acid sequence from a biological sample (e.g., from one or more biological samples) that is different from the biological samples used to generate sequencing data employed for the training of the model. Typically, the biological sample is from a subject that was not included in the population of subjects from which the plurality of sequencing data files that were used for training the model was collected. In some implementations, the nucleic acid sequence includes a deoxyribonucleic acid (DNA) sequence or a ribonucleic acid (RNA) sequence. In some implementations, the nucleic acid sequence includes a deoxyribonucleic acid (DNA) sequence. In some implementations, the nucleic acid sequence includes a sequence of at least 50 nucleotides (e.g., at least 75 nucleotides, at least 100 nucleotides, or at least 150 nucleotides). In some implementations, the sequencing of the nucleic acid sequence to obtain the sequencing reads includes paired end or single end sequencing. In some implementations, the sequencing of the nucleic acid sequence to obtain the sequencing reads includes whole genome sequencing or whole exome sequencing. In some instances, the sequencing of the nucleic acid sequence to obtain the sequencing reads includes somatic sequencing. In some instances, the sequencing of the nucleic acid sequence to obtain the sequencing reads includes germline sequencing.

The trained variant caller model 200 executes on the computing system 120 and/or the user computing device 110 to identify variants in a sequencing read 104 (e.g., a LP sequencing read) performed on a biological sample collected from a subject. As mentioned previously, a LP sequencing read includes sequencing of a nucleic acid sequence from the biological sample collected from the subject at a depth of 10× or less (e.g., at a depth of 10× or less, e.g., 4× or less, e.g., 0.1 to 4×).

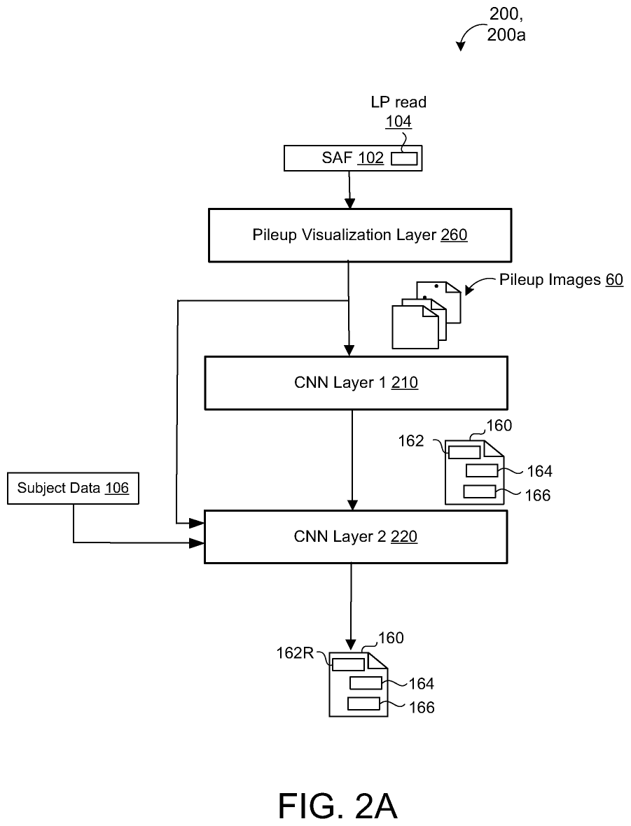

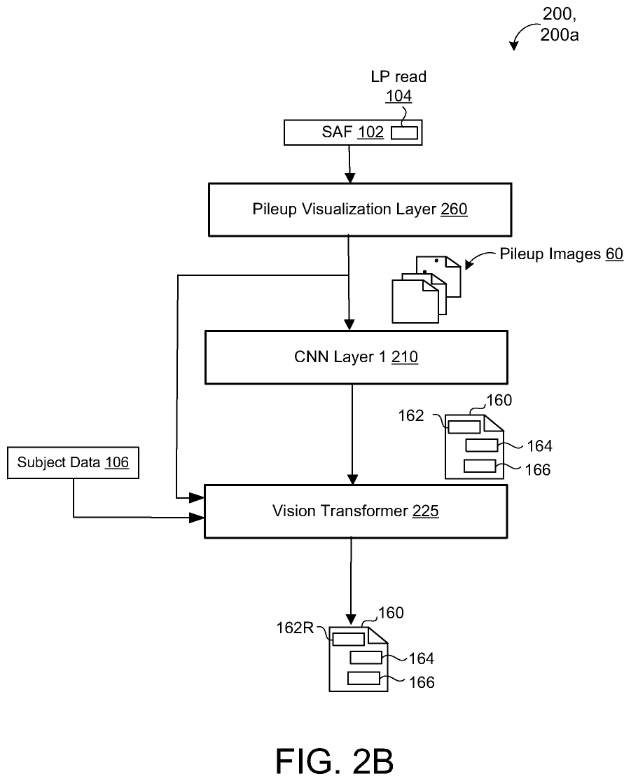

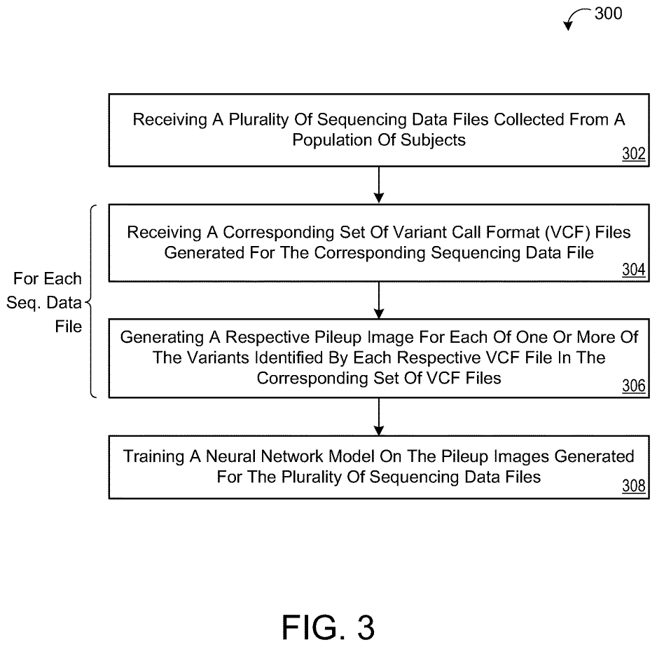

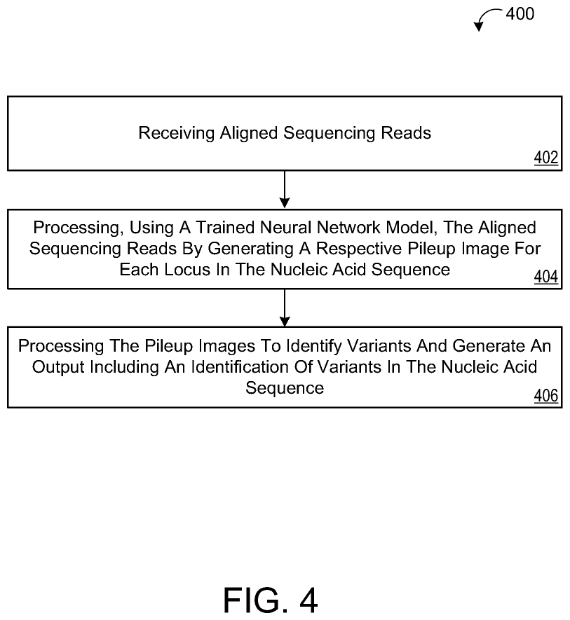

Here, the trained sequencing variant caller model 200 receives a sequence alignment file 102 (e.g., a BAM file or other type of sequencing alignment file) that stores the precise genomic locations or positions where each sequencing read in the nucleic acid sequence aligns to a reference genome. The trained sequencing variant caller model 200 processes the sequence alignment file 102 by: generating a respective pileup image 60 for each locus in the nucleic acid sequence; and processing the respective pileup image 60 generated for each locus (FIGS. 2A and 2B) to generate, as output, an identification of variants 162 in the nucleic acid sequence. Notably, the trained sequencing variant caller model 200 does not perform imputation based on information from a reference panel to identify variants.

In some examples, the output (e.g., VCF file) 160 generated by the trained sequencing variant caller model 200, in addition to identifying variants 162, further provides confidence scores 164, variant allele frequencies (VAFs) 166 and/or corresponding VAF confidence intervals 166 for variants (e.g., some or all of the variants) identified in the nucleic acid sequence. The corresponding confidence scores 164, VAFs 166 and/or VAF confidence intervals 166 may be stored as metadata 167 in the VCF file 160. The metadata 167 may include other pertinent information such as depth coverage, mapping quality, and/or other statistics that may be relevant to understanding the input and/or output of the trained model 200.

In some implementations, the trained sequencing variant caller model 200 also receives subject data 106 associated with the subject from which the biological was collected. In some such implementations, processing the pileup images 60 generated for each locus in the nucleic acid sequence further includes processing the pileup images 60 and the subject data 106 associated with the subject to generate the output 160. Such output 160 may include an identification of each variant 162 in the nucleic acid sequence; and optionally a corresponding confidence score 164, VAF 166, and/or VAF confidence intervals 166 for variants 162 identified in the nucleic acid sequence. As discussed above with respect to the training process 200, the subject data 106 may include EMR data 106a associated with the subject and/or sample data 106b associated with the biological sample collected from the subject.

The variants 162 included in the output 160 may include germline variant calls or somatic variant calls. The variants may include at least one of SNVs, insertions, or deletions. In some instances, the variants are SNVs.

With continued reference to FIG. 1, the output 160 from the trained sequencing variant caller model 200 may be provided to a treatment recommender 170. Here, the output 160 may be applicable to diagnose, prognosticate, or monitor disease progression of the subject from which the biological sample was collected. For example, the subject can be a human patient, such subject having or at risk for a disease (e.g., a cancer, an autoimmune disease, a cardiovascular disease, or a neurologic disease). In some scenarios, the output 160 provides information about whether or not the subject carries somatic variants that are implicated in certain diseases or conditions. Accordingly, the treatment recommender 170 may output a treatment recommendation 172 based on the output 160. Notably, the output 160 may help guide therapeutic intervention for the subject. Information about variants in nucleic acid sequences from a subject can be used for precision medicine approaches. For instance, the presence or absence of a variant may be directly or indirectly associated with an individual's responsiveness to a drug, the safety of a drug for an individual, or the dose of a drug that is likely to be safe and/or effective in an individual subject. In some instances, the treatment recommender will recommend or not recommend a particular treatment, or will provide statistical information or dosing recommendations (e.g., the dose or administration frequency of a drug that is likely to be safe and/or efficacious in the subject), based on output 160. In some instances, treatment recommender 170 calculates and provides as output a risk score (e.g., a polygenic risk score) or other probability score (e.g., a likelihood of responding to a treatment or a likelihood of an adverse response to a treatment) based on information included in output 160. In some instances, the treatment recommender 170 also receives and uses subject data 106 for the applicable subject to generate its output.

In some examples, multiple biological samples are collected from a respective subject at different timepoints and a respective LP sequencing read 104 is performed on the biological sample collected from the respective subject at each respective different timepoint. The LP sequencing reads 104 performed on the biological samples collected from the subject at the different timepoints may include somatic sequencing of a nucleic acid sequence from each biological sample. For instance, when the respective subject includes a subject diagnosed with cancer, the multiple biological samples collected from the respective subject may include tumor biopsy or liquid biopsy samples. For each of the multiple biological samples, a corresponding sequence alignment file (e.g., BAM file) 102 may be obtained that stores the precise genomic locations or positions where each sequencing read in the nucleic acid sequence aligns to a reference genome. Accordingly, for each of the multiple biological samples collected from the respective subject at different timepoints, the trained sequencing variant caller model 200 may be used to provide output 160 including an identification of variants in the nucleic acid sequence and, optionally corresponding confidence scores. The output 160 may be in the form of a VCF file. In this way, the tumor biopsy or liquid biopsy samples collected from the respective subject at different timepoints allow for monitoring disease progression using the trained sequencing variant caller model 200. The trained sequencing variant caller model 200 may be used for detecting the presence or absence of somatic variants at the different timepoints. In some scenarios, biological samples collected from the respective subject include samples such as, without limitation, blood samples, urine samples, sputum samples, or cerebrospinal fluid samples. In some scenarios, a presence or absence of a known somatic variant or appearance of a new somatic variant is correlated with tissue QC parameters that indicate different stages of progression. In some implementations, such QC parameters are included as subject data 106 that is input into the trained model 200. With respect to cancer, a number or amount of new mutations detected may increase as cancer progresses from early stage to late stage.

The treatment recommendation 172 output from the treatment recommender 170 may indicate a clinical action or decision based on an indication of diagnosis of a disease inferred from the information contained in the output 160. For instance, the treatment recommendation 172 may indicate a medication to prescribe to the subject, a recommendation for the subject to participate in a clinical trial, or a recommendation of a clinical test to confirm the diagnosis of the disease. The clinical test may include, without limitation, at least one of an imaging test, a blood test, a computed tomography (CT) scan, a magnetic resonance imaging (MRI) scan, an ultrasound scan, an X-ray, a positron emission tomography (PET) scan, or a cell-free biological cytology.

Referring to FIGS. 2A and 2B, the trained neural network model 200 may include at least one of a convolutional neural network (CNN) or a vision transformer (ViT). CNNs are a specialized type of neural network that can be transitionally invariant by their ability to learn to extract features regardless of their location. A network may be regarded as having a CNN architecture if, in at least one layer, a convolution instead of matrix multiplication is applied.

FIG. 2A provides an example of the trained neural network model 200 including a CNN multi-module architecture 200a that includes a pileup visualization layer 260, a first CNN layer 210, and a second CNN layer 220. The pileup visualization layer 260 is configured to receive, as input, aligned sequencing reads 104 obtained by sequencing a nucleic acid sequence at a depth of 10x or less from a biological sample 20 collected from a subject, and generate, as output, a respective pileup image 60 for each locus in the nucleic acid sequence. The aligned sequencing reads 104 may include a sequence alignment file 102. For instance, the sequence alignment file 102 may include a BAM file.

The first CNN layer 210 is configured to receive, as input from the pileup visualization layer 260, the respective pileup image 60 generated for each locus in the nucleic acid sequence, and generate, as output, an output 160 indicating an identification of variants 162 in the nucleic acid sequence. In some examples, the output 160 generated as output from the first CNN layer 210 further includes at least one of corresponding confidence scores 164, corresponding VAFs 166, and/or corresponding VAF confidence intervals 166 for the variants included in the identification of variants 162 in the nucleic acid sequence. In some scenarios, the trained neural network model 200 only includes the pileup visualization layer 260 and the first CNN layer 210 for generating the output 160.

The second CNN layer 220 is configured to: receive, as input, subject data 106 associated with the subject and at least one of the output 160 of the first CNN layer 210 or the respective pileup image 60 for each locus in the nucleic acid sequence generated as output from the pileup visualization layer 260; and generate, as output, a refined output 160R including a refined identification of variants in the nucleic acid sequence. In some examples, the refined output 160R generated as output from the second CNN layer 220 further includes at least one of corresponding confidence scores 164, corresponding VAFs 166, and/or corresponding VAF confidence intervals 166 for the variants included in the refined identification of variants 162R in the nucleic acid sequence.

In some scenarios, the trained neural network model 200 only includes the pileup visualization layer 260 and the first CNN layer 210 such that the output 160 from the trained neural network model 200 in FIG. 1 includes the output 160 generated by the first CNN layer 210. In other scenarios, the trained network model 200 includes the pileup visualization layer 260, the first CNN layer 210, and the second CNN layer 220 such that the output 160 from the trained neural network model 200 in FIG. 1 includes the revised output 160 generated by the second CNN layer 220.

FIG. 2B provides an example of the trained neural network model 200 including another multi-module architecture 200b that includes a pileup visualization layer 260, a first CNN layer 210, and a ViT 225. Here, the functionality is substantially similar to the architecture 200a of FIG. 2A except that the second CNN layer 220 is replaced by the ViT 225. For instance, the ViT 225 may be configured to: receive, as input, subject data 106 associated with the subject and at least one of the output 160 of the first CNN layer 210 or the respective pileup image 60 for each locus in the nucleic acid sequence generated as output from the pileup visualization layer 260; and generate, as output, a refined output 160R including a refined identification of variants in the nucleic acid sequence.

The example architectures of the trained neural network model 200 are exemplary only and may include additional CNN layers without departing from the scope of the present disclosure. For instance, a third CNN layer may receive sample data 106a such as methylation profiling data associated with the biological sample in order to provide deeper sensitivity beyond the level of variants that the trained neural network model can detect. The methylation profiling data ingested by the additional CNN layer may include methylation reads that sequence methylation patterns/markers after DNA from the biological sample is modified using methylation-sensitive enzymes/chemicals/agents to distinguish between methylated and un-methylated cytosines in the DNA. The methylation profiling data may sequence the methylation patterns/markers from a different biological sample from the subject than the biological sample 20 associated with the LP sequencing read 104. For instance, the biological sample 20 associated with the LP sequencing read 104 may include a tissue sample biopsied from the subject while the biological sample associated with the methylation profiling may include a liquid biopsy collected from the same subject. In fact, the methylation profiling data may sequence corresponding patterns/markers of methylation from biological samples collected at different times. Hence, as collection of liquid biopsies is non-invasive, the methylation profiling data may include include: methylation patterns/markers from a first liquid biopsy collected from the subject at a first time; and methylation patterns/markers from a second liquid biopsy collected from the subject at a second time after the first time.

By contrast to tumor tissue samples, liquid biopsies (e.g., blood) are limited on the amount of circulating tumor DNA. Therefore, the training of the additional CNN layer to ingest methylation profiling data indicating methylation patterns/markers within liquid biopsies provides additional biological signals for confidently detecting/predicting various outputs. The outputs may include, without limitation, disease detection/prognosis, disease resistant states, and response to therapeutics or therapies.

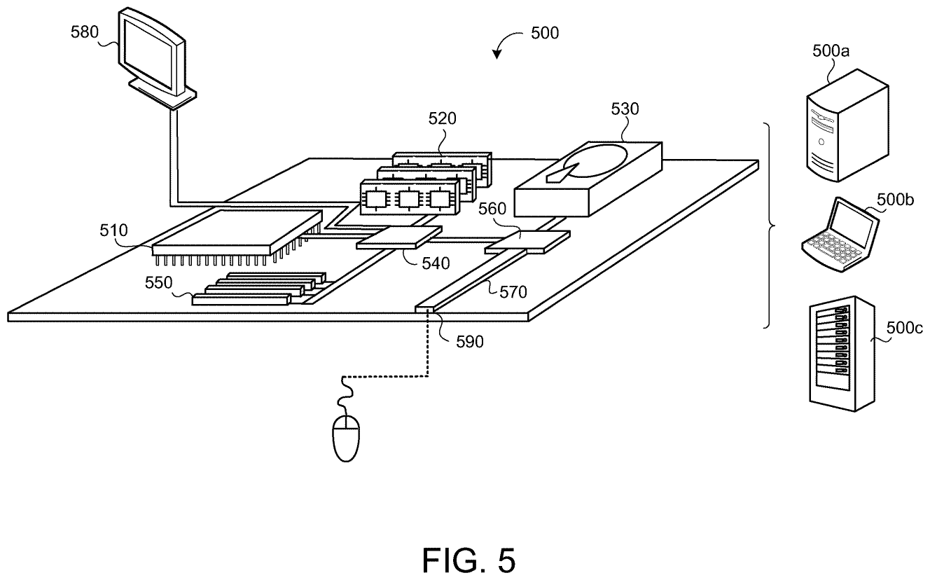

FIG. 3 is a flowchart of an example arrangement of operations for a method 300 of training a neural network model 200 on pileup images 60 generated for a plurality of sequencing data files 10 to teach the neural network model 200 to learn how to generate output variant calls from sequencing data inputs obtained from sequencing nucleic acid sequences at depths of 10x or less from biological samples. The method 300 may execute on the data processing hardware 122 of the computing system 120 based on instructions stored on the memory hardware 124 of the computing system 120. The computing system 120 may include computing device 500 (FIG. 5), the data processing hardware 122 may include data processing hardware 510 (FIG. 5), and the memory hardware 124 may include memory hardware 520 (FIG. 5). At operation 302, the method 300 includes receiving a plurality of sequencing data files 10 collected from a population of subjects. Here, each sequencing data file 10 is obtained from sequencing a nucleic acid sequence from a corresponding biological sample 20 collected from a corresponding subject in the population of subjects.

For each corresponding sequencing data file 10 among the plurality of sequencing data files 10, the method 300 performs operations 304 and 306. At operation 304, the method 300 includes receiving a corresponding set of variant call format (VCF) files 50 generated for the corresponding sequencing data file 10. Here, each respective VCF file 50 in the corresponding set of VCF files 50 is generated as output from a respective one of a plurality of independent variant caller applications 250 that receive the corresponding sequencing data file 10 as input. Moreover, each respective VCF file 50 in the corresponding set of VCF files 50 identifies variants. At operation 306, the method 300 includes generating a respective pileup image 60 for each of one or more of the variants identified by each respective VCF file 50 in the corresponding set of VCF files 50.

At operation 308, the method 300 includes training the neural network model 200 on the pileup images 60 generated for the plurality of sequencing data files 10 to teach the neural network model 200 to learn how to generate an output 160 that identifies variants 162 from sequencing data inputs obtained from sequencing nucleic acid sequences at depths of 10× or less from one or more other biological samples. The neural network model 200 may include at least one of a vision transformer (ViT) or a convolutional neural network (CNN). Training the neural network model 200 on the pileup images 60 may further teach the neural network model 200 to learn how to generate corresponding confidence scores 164, corresponding variant allele frequencies (VAFs) 166, and/or corresponding VAF confidence intervals 166 for the variants 162 identified in the output 160.

FIG. 4 is a flowchart of an example arrangement of operations for a method 400 of using a trained neural network model 200 to identify variants in a nucleic acid sequence based on aligned sequencing reads 102 obtained by sequencing the nucleic acid sequence at a depth of 10× or less from a biological sample collected from a subject. The trained neural network model 200 may be trained using the training process 150 of FIG. 1. The method 400 may execute on data processing hardware 510 (FIG. 5) implemented on a computing device 500 (FIG. 5) (e.g., user computing device 110 and/or computing system 120 of FIG. 1) based on instructed stored on memory hardware 520 (FIG. 5).

At operation 402, the method 400 includes receiving aligned sequencing reads 102. Here, the sequencing reads 102 are obtained by sequencing a nucleic acid sequence at a depth of 10× or less from a biological sample collected from a subject. At operation 404, the method 400 includes processing, using the trained neural network model 200, the aligned sequencing reads 102 by generating a respective pileup image 60 for each locus in the nucleic acid sequence. At operation 406, the method 400 includes processing the pileup images generated for each locus in the nucleic acid sequence to identify variants and generate an output 160 including an identification of variants 162 in the nucleic acid sequence. The output 160 generated by processing the pileup images 60 may further include at least one of corresponding confidence scores 164, corresponding VAFs 166, and/or corresponding VAF confidence intervals 166 for the variants included in the identification of variants 162 in the nucleic acid sequence.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

The non-transitory memory may be physical devices used to store programs (e.g., sequences of instructions) or data (e.g., program state information) on a temporary or permanent basis for use by a computing device. The non-transitory memory may be volatile and/or non-volatile addressable semiconductor memory. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

FIG. 5 is schematic view of an example computing device 500 that may be used to implement the systems and methods described in this document. The computing device 500 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 500 includes a processor 510, memory 520, a storage device 530, a high-speed interface/controller 540 connecting to the memory 520 and high-speed expansion ports 550, and a low speed interface/controller 560 connecting to a low speed bus 570 and a storage device 530. Each of the components 510, 520, 530, 540, 550, and 560, are interconnected using various busses, and may be mounted on a common motherboard or in other manners as appropriate. The processor 510 (i.e., data processing hardware) can process instructions for execution within the computing device 500, including instructions stored in the memory 520 (i.e., memory hardware) or on the storage device 530 to display graphical information for a graphical user interface (GUI) on an external input/output device, such as display 580 coupled to high speed interface 540. In other implementations, multiple processors and/or multiple buses may be used, as appropriate, along with multiple memories and types of memory. Also, multiple computing devices 500 may be connected, with each device providing portions of the necessary operations (e.g., as a server bank, a group of blade servers, or a multi-processor system).

The memory 520 stores information non-transitorily within the computing device 500. The memory 520 may be a computer-readable medium, a volatile memory unit(s), or non-volatile memory unit(s). The non-transitory memory 520 may be physical devices used to store programs (e.g., sequences of instructions) or data (e.g., program state information) on a temporary or permanent basis for use by the computing device 500. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 530 is capable of providing mass storage for the computing device 500. In some implementations, the storage device 530 is a computer-readable medium. In various different implementations, the storage device 530 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer-or machine-readable medium, such as the memory 520, the storage device 530, or memory on processor 510.

The high speed controller 540 manages bandwidth-intensive operations for the computing device 500, while the low speed controller 560 manages lower bandwidth-intensive operations. Such allocation of duties is exemplary only. In some implementations, the high-speed controller 540 is coupled to the memory 520, the display 580 (e.g., through a graphics processor or accelerator), and to the high-speed expansion ports 550, which may accept various expansion cards (not shown). In some implementations, the low-speed controller 560 is coupled to the storage device 530 and a low-speed expansion port 590. The low-speed expansion port 590, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 500 may be implemented in a number of different forms, as shown in the figure. For example, it may be implemented as a standard server 500a or multiple times in a group of such servers 500a, as a laptop computer 500b, or as part of a rack server system 500c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims.

1-128. (canceled)

129. A computer-implemented method executing on data processing hardware that causes the data processing hardware to perform operations comprising:
receiving a plurality of sequencing data files collected from a population of subjects, each sequencing data file obtained from sequencing a nucleic acid sequence from a corresponding biological sample collected from a corresponding subject in the population of subjects;
for each corresponding sequencing data file among the plurality of sequencing data files:
receiving a corresponding set of variant call format (VCF) files generated for the corresponding sequencing data file, each respective VCF file in the corresponding set of VCF files generated as output from a respective one of a plurality of independent variant caller applications that receive the corresponding sequencing data file as input, each respective VCF file in the corresponding set of VCF files identifying variants; and
for each of one or more of the variants identified by each respective VCF file in the corresponding set of VCF files, generating a respective pileup image; and
training a neural network model on the pileup images generated for the plurality of sequencing data files to teach the neural network model to learn how to generate an output identifying variants from sequencing data inputs obtained from sequencing nucleic acid sequences at depths of 10× or less from one or more other biological samples.

130. The computer-implemented method of claim 129, wherein the corresponding sequencing data file comprises a sequence alignment file (SAF) obtained from the sequencing of the nucleic acid sequence from the corresponding biological sample.

131. The computer-implemented method of claim 129, wherein the nucleic acid sequence comprises a deoxyribonucleic acid (DNA) sequence or a ribonucleic acid (RNA) sequence.

132. The computer-implemented method of claim 129, wherein the sequencing of the nucleic acid sequence from at least one of the plurality of biological samples is performed using whole genome sequencing and the sequencing of the nucleic acid sequence from at least another one of the plurality of biological samples is performed using whole exome sequencing.

133. The computer-implemented method of claim 129, wherein the plurality of biological samples collected from the population of subjects comprises a subset of biological samples collected from a respective subject in the population of subjects.

134. The computer-implemented method of claim 133, wherein:
for at least one corresponding biological sample from the subset of biological samples collected from the respective subject, the sequencing of the nucleic acid sequence from the at least one corresponding biological sample comprises somatic sequencing of the nucleic acid sequence from the at least one corresponding biological sample from the subset of biological samples collected from the respective subject; and
for at least another corresponding biological sample from the subset of biological samples collected from the respective subject, the sequencing of the nucleic acid sequence from the at least another corresponding biological sample comprises germline sequencing of the nucleic acid sequence from the at least another corresponding biological sample from the subset of biological samples collected from the respective subject.

135. The computer-implemented method of claim 133, wherein two or more biological samples in the subset of biological samples collected from the respective subject are collected at different timepoints.

136. The computer-implemented method of claim 129, wherein the corresponding sequencing data file comprises one or more quality control (QC) parameters for one or more loci in the nucleic acid sequence sequenced from the biological samples.

137. The computer-implemented method of claim 136, wherein the operations further comprise, for the variants identified by the corresponding set of VCF files generated for the corresponding sequencing data file:
filtering the variants based on the one or more QC parameters of the corresponding sequencing data file to remove any variants in the corresponding set of VCF files that are artifacts,
wherein generating the respective pileup image for each of the one or more of the variants identified by each respective VCF file in the corresponding set of VCF files comprises generating the respective pileup image for each of the one or more of the variants after filtering to remove the variants in the corresponding set of VCF files that are artifacts.

138. The computer-implemented method of claim 129, wherein the variants identified by each respective VCF file in the corresponding set of VCF files comprise at least one of single nucleotide variants (SNVs), insertions, or deletions.

139. The computer-implemented method of claim 129, wherein the operations further comprise, for at least one corresponding sequencing data file:
obtaining a corresponding benchmark data set of variant calls for the nucleic acid sequence sequenced from the corresponding biological sample; and
for each respective VCF file in the corresponding set of VCF files generated for the corresponding sequencing data file, annotating, using the benchmark data set of variant calls, the respective pileup image generated for each of the one or more of the variants identified by the respective VCF file to flag:
any variants correctly identified by the respective VCF file;
any variants incorrectly identified by the respective VCF file; and
any variants unidentified by the respective VCF file,
wherein training the neural network model on the pileup images comprises training the neural network on the annotated pileup images.

140. The computer-implemented method of claim 129, wherein training the neural network model further comprises training the neural network model on the pileup images to teach the neural network model to learn how to generate corresponding confidence scores for the variants identified in the output.

141. The computer-implemented method of claim 129, wherein training the neural network model further comprises training the neural network model on the pileup images to teach the neural network model to learn how to generate corresponding variant allele frequencies (VAFs) and/or corresponding VAF confidence intervals for the variants identified in the output.

142. The computer-implemented method of claim 129, wherein the neural network model is trained on the pileup images to teach the neural network model to learn how to generate the output identifying variants without imputation based on information from a reference panel.

143. The computer-implemented method of claim 129, wherein the operations further comprise:
obtaining a test sequencing data set comprising:
a plurality of testing sequencing data inputs each obtained by sequencing a corresponding nucleic acid sequence from a corresponding test sample; and
for each corresponding testing sequencing data input, a corresponding benchmark data set of variant calls for the corresponding nucleic acid sequence; and
after training the neural network model, for each testing sequence data input among the plurality of testing sequencing data inputs of the sequencing data test set:
processing, using the trained neural network model, the testing sequence data input to predict output variant calls identified in the corresponding nucleic acid sequence; and
assessing, using one or more metrics, the predicted output variant calls based on the corresponding benchmark data set of variant calls for the corresponding nucleic acid sequence.

144. A system comprising:
data processing hardware; and
memory hardware in communication with the data processing hardware and storing instructions that when executed by the data processing hardware causes the data processing hardware to perform operations comprising:
receiving a plurality of sequencing data files collected from a population of subjects, each sequencing data file obtained from sequencing a nucleic acid sequence from a corresponding biological sample collected from a corresponding subject in the population of subjects;
for each corresponding sequencing data file among the plurality of sequencing data files:
receiving a corresponding set of variant call format (VCF) files generated for the corresponding sequencing data file, each respective VCF file in the corresponding set of VCF files generated as output from a respective one of a plurality of independent variant caller applications that receive the corresponding sequencing data file as input, each respective VCF file in the corresponding set of VCF files identifying variants; and
for each of one or more of the variants identified by each respective VCF file in the corresponding set of VCF files, generating a respective pileup image; and
training a neural network model on the pileup images generated for the plurality of sequencing data files to teach the neural network model to learn how to generate an output identifying variants from sequencing data inputs obtained from sequencing nucleic acid sequences at depths of 10× or less from one or more other biological samples.

145. The system of claim 144, wherein the corresponding sequencing data file comprises a sequence alignment file (SAF) obtained from the sequencing of the nucleic acid sequence from the corresponding biological sample.

146. The system of claim 144, wherein the nucleic acid sequence comprises a deoxyribonucleic acid (DNA) sequence or a ribonucleic acid (RNA) sequence.

147. The system of claim 144, wherein the sequencing of the nucleic acid sequence from at least one of the plurality of biological samples is performed using whole genome sequencing and the sequencing of the nucleic acid sequence from at least another one of the plurality of biological samples is performed using whole exome sequencing.

148. The system of claim 144, wherein the plurality of biological samples collected from the population of subjects comprises a subset of biological samples collected from a respective subject in the population of subjects.

149. The system of claim 148, wherein:
for at least one corresponding biological sample from the subset of biological samples collected from the respective subject, the sequencing of the nucleic acid sequence from the at least one corresponding biological sample comprises somatic sequencing of the nucleic acid sequence from the at least one corresponding biological sample from the subset of biological samples collected from the respective subject; and
for at least another corresponding biological sample from the subset of biological samples collected from the respective subject, the sequencing of the nucleic acid sequence from the at least another corresponding biological sample comprises germline sequencing of the nucleic acid sequence from the at least another corresponding biological sample from the subset of biological samples collected from the respective subject.

150. The system of claim 148, wherein two or more biological samples in the subset of biological samples collected from the respective subject are collected at different timepoints.

151. The system of claim 144, wherein the corresponding sequencing data file comprises one or more quality control (QC) parameters for one or more loci in the nucleic acid sequence sequenced from the biological samples.

152. The system of claim 151, wherein the operations further comprise, for the variants identified by the corresponding set of VCF files generated for the corresponding sequencing data file:
filtering the variants based on the one or more QC parameters of the corresponding sequencing data file to remove any variants in the corresponding set of VCF files that are artifacts,
wherein generating the respective pileup image for each of the one or more of the variants identified by each respective VCF file in the corresponding set of VCF files comprises generating the respective pileup image for each of the one or more of the variants after filtering to remove the variants in the corresponding set of VCF files that are artifacts.

153. The system of claim 144, wherein the variants identified by each respective VCF file in the corresponding set of VCF files comprise at least one of single nucleotide variants (SNVs), insertions, or deletions.

154. The system of claim 144, wherein the operations further comprise, for at least one corresponding sequencing data file:
obtaining a corresponding benchmark data set of variant calls for the nucleic acid sequence sequenced from the corresponding biological sample; and
for each respective VCF file in the corresponding set of VCF files generated for the corresponding sequencing data file, annotating, using the benchmark data set of variant calls, the respective pileup image generated for each of the one or more of the variants identified by the respective VCF file to flag:
any variants correctly identified by the respective VCF file;
any variants incorrectly identified by the respective VCF file; and
any variants unidentified by the respective VCF file,
wherein training the neural network model on the pileup images comprises training the neural network on the annotated pileup images.

155. The system of claim 144, wherein training the neural network model further comprises training the neural network model on the pileup images to teach the neural network model to learn how to generate corresponding confidence scores for the variants identified in the output.

156. The system of claim 144, wherein training the neural network model further comprises training the neural network model on the pileup images to teach the neural network model to learn how to generate corresponding variant allele frequencies (VAFs) and/or corresponding VAF confidence intervals for the variants identified in the output.

157. The system of claim 144, wherein the neural network model is trained on the pileup images to teach the neural network model to learn how to generate the output identifying variants without imputation based on information from a reference panel.

158. The system of claim 144, wherein the operations further comprise:
obtaining a test sequencing data set comprising:
a plurality of testing sequencing data inputs each obtained by sequencing a corresponding nucleic acid sequence from a corresponding test sample; and
for each corresponding testing sequencing data input, a corresponding benchmark data set of variant calls for the corresponding nucleic acid sequence; and
after training the neural network model, for each testing sequence data input among the plurality of testing sequencing data inputs of the sequencing data test set:
processing, using the trained neural network model, the testing sequence data input to predict output variant calls identified in the corresponding nucleic acid sequence; and
assessing, using one or more metrics, the predicted output variant calls based on the corresponding benchmark data set of variant calls for the corresponding nucleic acid sequence.
